(19) United States
(12) Patent Application Publication
(10) Pub. No.: US 2025/0132056 A1
(43) Pub. Date: Apr. 24, 2025

(54) POPULATION SPECIFIC RADIOMIC IMAGING BIOMARKERS ASSOCIATED WITH CLINICALLY SIGNIFICANT CANCER

(71) Applicants: Emory University, Atlanta, GA (US); University Hospitals Cleveland Medical Center, Cleveland, OH (US)

(72) Inventors: Rakesh Shiradkar, Decatur, GA (US); Abhishek Midya, New York, NY (US); Anant Madabhushi, Decatur, GA (US); Lee E. Ponsky, Cleveland, OH (US)

(21) Appl. No.: 18/925,674

(22) Filed: Oct. 24, 2024

Related U.S. Application Data
(60) Provisional application No. 63/592,707, filed on Oct. 24, 2023.

(51) Int. Cl. G16H 50/70; G16H 30/40; G16H 50/30

(52) U.S. Cl. CPC G16H 50/70; G16H 30/40; G16H 50/30

(57) ABSTRACT

The present disclosure, in some embodiments, relates to a method. The method includes accessing one or more digitized images of a cancer patient of a first population. One or more regions of interest are identified within the one or more digitized images. A plurality of population specific features are extracted from the one or more regions of interest within the one or more digitized images. The plurality of population specific features are features that have been identified as being prognostic of an outcome for patients of the first population. A population specific machine learning model is operated upon the plurality of population specific features to generate a medical prediction relating to the outcome.

### REFERENCE TO RELATED APPLICATION

This Application claims the benefit of U.S. Provisional Application No. 63/592,707, filed on Oct. 24, 2023, the contents of which are incorporated by reference in their entirety.

### BACKGROUND

In recent years, there has been significant interest in developing machine vision tools for interrogating medical images. Machine vision tools are computer systems that utilize artificial intelligence to analyze medical images. Such systems have the potential to improve health care for patients.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a block diagram of machine vision system comprising population specific machine learning models configured to determine a medical prediction for a cancer patient.

FIG. 2 illustrates some embodiments of a flow diagram showing a method for using population specific machine learning models to determine medical predictions for cancer patients.

FIG. 3 illustrates some additional embodiments of a block diagram of a machine vision system comprising population specific machine learning models configured to determine a medical prediction for a cancer patient.

FIG. 4 illustrates some additional embodiments of a block diagram of a machine vision system comprising population specific machine learning models configured to determine a medical prediction for a cancer patient.

FIGS. 5A-5D illustrate exemplary images showing radiomic features within one or more regions of interest (ROI) corresponding to patients of different populations classified as having clinically significant cancer and clinically insignificant cancer.

FIG. 6 illustrates some additional embodiments of a block diagram of a prostate cancer assessment system comprising population specific machine learning models configured to determine a population specific risk score for a prostate cancer patient.

FIG. 7A shows exemplary violin plots of radiomic features from Magnetic Resonance Imaging (MRI) associated with clinically significant prostate cancer in African American patients and Caucasian American patients.

FIG. 7B shows a graph illustrating exemplary receiver operating characteristic (ROC) curves demonstrating the classification performance of population specific machine learning models for African American patients and Caucasian American patients.

FIG. 8 illustrates a table showing some exemplary radiomic features that a disclosed machine vision system may extract from tumoral regions and peri-tumoral regions of images from a cancer patient.

FIG. 9 illustrates a block diagram of some additional embodiments of a machine vision system comprising population specific machine learning models that are configured to be trained to determine a medical prediction for a patient using population specific features extracted from digitized images.

FIG. 10 illustrates a block diagram showing an example patient selection for a disclosed method of training a disclosed machine vision system.

FIG. 11 illustrates a table showing some exemplary performance metrics achieved by different biomarkers used to predict prostate cancer risk.

FIG. 12 illustrates a block diagram of some embodiments of a prognostic apparatus comprising a disclosed machine vision system configured to use population specific machine learning models to generate a medical prediction.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Cancer refers to any one of a large number of diseases characterized by the development of abnormal cells that divide uncontrollably and have the ability to infiltrate and destroy normal body tissue. Over time, cancer may spread throughout a person's body. Cancer is the second-leading cause of death in the world. Prostate cancer is one of the most common types of cancer among men. Prostate cancer begins within cells in the prostate gland (e.g., a part of the reproductive system in men below the bladder). The most common risk factors for prostate cancer include age, race, and family history. For example, the risk of developing prostate cancer increases as you get older, so that nearly 60% of prostate cancer patients are over the age of 65.

It has been appreciated that some cancers may disproportionately affect different population groups. For example, the risk of developing prostate cancer is higher (e.g., approximately 1.5 times higher) for African American men (e.g., men that are black and/or of African ancestry) than for Caucasian American men. African American men also have a greater risk of developing prostate cancers that are likely to spread, have a greater risk of developing prostate cancer before age 50, and are 2.2 times more likely to die from prostate cancer than Caucasian American men. The higher risks may be due to genetic mutations, thereby suggesting a biological and/or genetic basis of the racial disparities.

In some embodiments, the present disclosure relates a method and associated apparatus for stratifying cancer risk using population specific models that account for biological and/or genetic differences between specific populations (e.g., between African American men and Caucasian American men on prostate imaging (MRI)). In some embodiments, the method may be performed by accessing a radiological image of a cancer patient (e.g., a prostate cancer patient). The cancer patient belongs to a first population (e.g., African American men, Caucasian American men, etc.). One or more regions of interest (ROI) are identified within the radiological image and a plurality of population specific features are extracted from within the one or more ROI. The plurality of population specific features are features that have been identified as being prognostic of cancer risk stratification within patients of the first population. The plurality of population specific features are operated upon by a population specific machine learning model to generate a population specific medical prediction. By operating a population specific machine learning model on population specific features, the disclosed method and apparatus are able to account for differences between different populations (e.g., between African American men and Caucasian American men). By accounting for differences between different populations, the disclosed method and apparatus are able to generate a medical prediction with a significantly higher accuracy than population agnostic models.

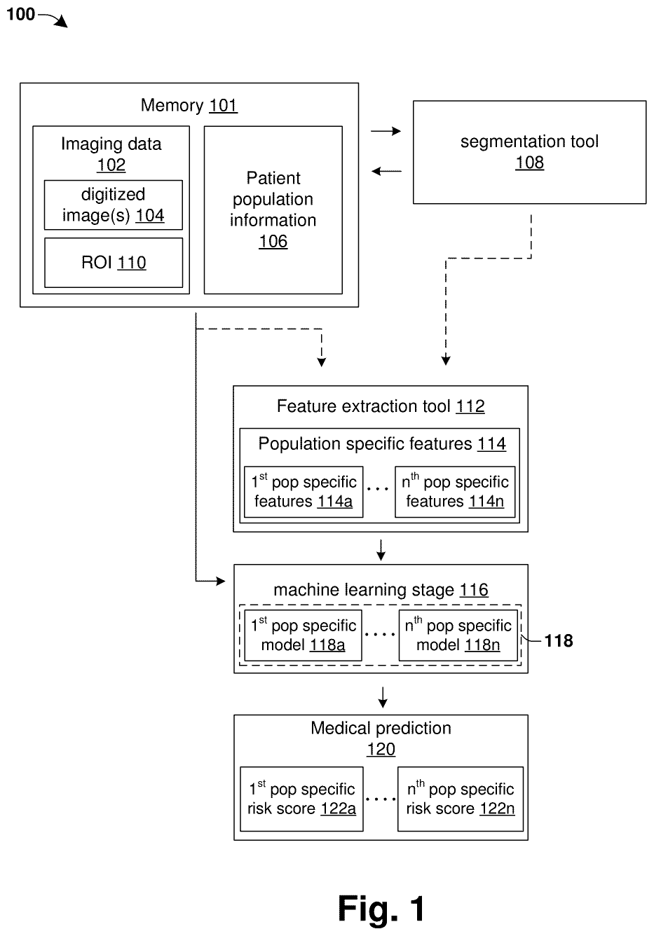

FIG. 1 illustrates some embodiments of a block diagram of machine vision system 100 comprising population specific machine learning models configured to determine a medical prediction for a cancer patient.

The machine vision system 100 comprises a memory 101 configured to store imaging data 102 from one or more cancer patients. In various embodiments, the one or more cancer patients may comprise a prostate cancer patient, a lung cancer patient, a breast cancer patient, an ovarian cancer patient, a gastrointestinal cancer patient, and/or the like. In some embodiments, the imaging data 102 may comprise one or more digitized images 104 (e.g., radiological images). In some embodiments, the imaging data 102 may comprise multi-parametric MRI imaging data, bi-parametric MRI imaging data, and/or the like.

The one or more digitized images 104 respectively have patient population information 106 associated with them. The patient population information 106 may describe groups of people that are of a same self-reported race, share physical characteristics (e.g., skin color, facial features, etc.), have same sexes (e.g., genders assigned at birth), have similar a genetic ethnicity and/or genetic ancestry, and/or the like. For example, the patient population information 106 may classify people as being African American men, African American women, Caucasian American men Caucasian American women, Hispanic American men, Hispanic American women, American Indian men, American Indian women, Asian American men, or the like.

In some embodiments, a segmentation tool 108 may be configured to segment the one or more digitized images 104 to identify one or more regions of interest (ROI) 110 within respective ones of the one or more digitized images 104. In some embodiments, the one or more segmented images and/or the one or more ROI 110 may be stored in the memory 101 as part of the imaging data 102. In other embodiments, the one or more segmented images and/or the one or more ROI 110 may be provided directly to a downstream tool.

A feature extraction tool 112 is configured to operate upon the imaging data 102 to extract a plurality of population specific features 114 from the one or more ROI 110. The plurality of population specific features 114 are radiomic features and/or imaging biomarkers that have been identified as being prognostic of an outcome for patients within a specific population. For example, the plurality of population specific features 114 may be radiomic features that have been identified as being prognostic of cancer risk stratification (e.g., that exhibit differences between clinically significant cancer and clinically insignificant cancer) for a patient within a specific population. In some embodiments, the feature extraction tool 112 may be configured to access the patient population information 106 associated with a first one of the one or more digitized images 104. Based upon the patient population information 106, the feature extraction tool 112 may be configured to extract either a first plurality of population specific features 114a that are prognostic of cancer risk stratification in a patient of a first population (e.g., African American) or a second plurality of population specific features 114n that are prognostic of cancer risk stratification in a patient of a second population (e.g., Caucasian American). The first plurality of population specific features 114a are different than the second plurality of population specific features 114n. In some embodiments, the plurality of population specific features 114 may comprise radiomic features that quantify heterogeneity associated with a cancerous tumor. For example, the plurality of population specific features 114 may comprise radiomic texture features (e.g., gray-level statistical features, Gabor features, Harlick features, Sobel features, etc.).

A machine learning stage 116 comprises a plurality of population specific machine learning models 118. In various embodiments, the plurality of population specific machine learning models 118 may comprise a first population specific machine learning model 118a and a second population specific machine learning model 118n. The machine learning stage 116 is configured to selectively operate one of the plurality of population specific machine learning models 118 upon the plurality of population specific features 114 to generate a medical prediction 120 relating to the outcome. In some embodiments, the medical prediction 120 may comprise a population specific risk score that stratifies a cancer risk for the cancer patient.

For example, the first population specific machine learning model 118a may operate upon the first plurality of population specific features 114a to generate a first population specific risk score 122a for patients of a first population, while the second population specific machine learning model 116n may operate upon the second plurality of population specific features 114n to generate a second population specific risk score 122n for patients of a second population. In some embodiments, the machine learning stage 116 may access the patient population information 106 to select which one of the plurality of population specific machine learning models 118a-118n is operated upon the plurality of population specific features 114.

By operating one of the plurality of population specific machine learning models 118 on the plurality of population specific features 114, the disclosed machine vision system 100 is able to generate the medical prediction 120 to account for biological and/or genetic differences between different populations (e.g., biological and/or genetic between African American men and Caucasian American men). By accounting for biological and/or genetic differences between different populations, the medical prediction 120 is able to achieve a significantly higher accuracy than medical predictions generated by population agnostic models (e.g., models that are not specific to a certain population, but rather apply to all populations). For example, a machine learning model that is configured to generate a population specific risk score relating to prostate cancer in African American men can achieve an AUC of approximately 0.80 in comparison to an AUC of approximately 0.55 achieved by population agnostic models. Furthermore, because radiological imaging is a routine standard of care, the disclosed machine vision system is able to account for genetic discrepancies between different patient population at a relatively low cost and with wide geographic availability (e.g., in comparison to genetic testing methods). The wide geographic availability may be significant to populations that are in geographic areas that do not have access to state of the art medical technologies.

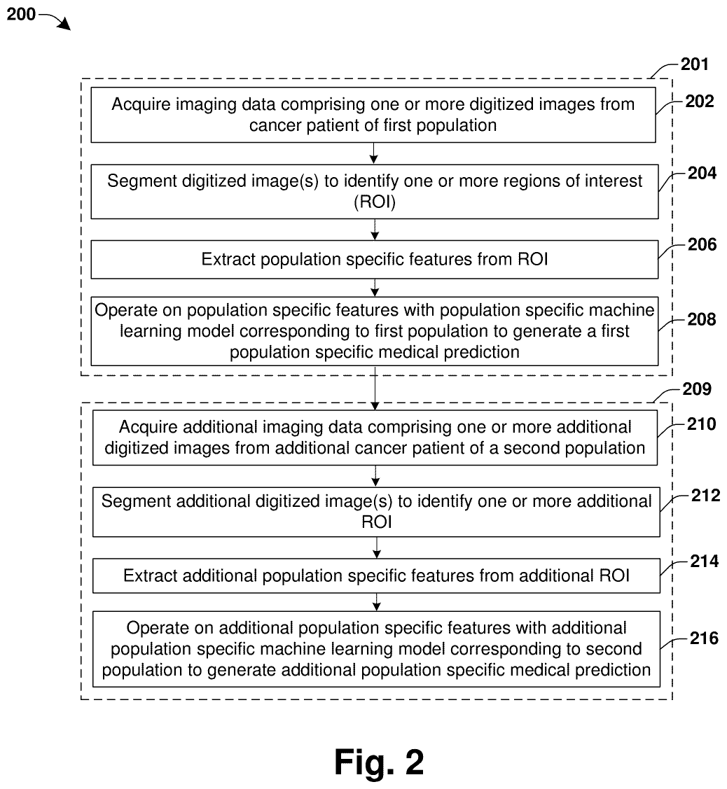

FIG. 2 illustrates some embodiments of a flow diagram showing a method 200 for using population specific machine learning models to determine medical predictions for cancer patients.

While the disclosed method 200 is illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 202, imaging data comprising one or more digitized images is acquired for a cancer patient of a first population. In some embodiments, the first population may be African American men. In some embodiments, the cancer patient may have prostate cancer. In some embodiments, the imaging data may comprise bi-parametric MRI imaging data.

At act 204, the one or more digitized images are segmented to identify one or more regions of interest (ROI). In some embodiments, the one or more ROI may comprise one or more of a tumoral region and a peri-tumoral region.

At act 206, a plurality of population specific features are extracted from the one or more ROI. In some embodiments, the plurality of population specific features may comprise texture features.

At act 208, the plurality of population specific features are operated upon by a population specific machine learning model corresponding to the first population to generate a population specific medical prediction for the cancer patient. In some embodiments, the population specific medical prediction may comprise a risk score that indicates a probability of a presence of clinically significant cancer within the cancer patient.

It will be appreciated that the disclosed method 200 may be applied to different patients belonging to different patient populations at different times. For example, in some embodiments, the method may be operated in a first instance 201 on a cancer patient of a first population (e.g., African American men) and in a second instance 209 on an additional cancer patient of a second population (e.g., Caucasian American men). In some embodiments, the first instance 201 may be performed according to acts 202-208, while the second instance 209 may be performed according to acts 210-216.

At act 210, additional imaging data comprising one or more additional digitized images is acquired for an additional cancer patient of a second population. In some embodiments, the second population may comprise Caucasian American men. In some embodiments, the additional cancer patient may have prostate cancer.

At act 212, the one or more additional digitized images are segmented to identify one or more additional ROI. In some embodiments, the one or more additional ROI may comprise one or more of a tumoral region and a peri-tumoral region.

At act 214, a plurality of additional population specific features are extracted from the one or more additional ROI. In some embodiments, the plurality of additional population specific features may comprise Haralick features.

At act 216, the plurality of additional population specific features are operated upon by an additional population specific machine learning model corresponding to the second population to generate an additional population specific medical prediction for the additional cancer patient. In some embodiments, the additional population specific medical prediction may comprise an additional population specific risk score that indicates a probability of a presence of clinically significant cancer within the additional cancer patient.

Therefore, the disclosed method 200 utilizes features and machine learning models that are specific to a population to generate a medical prediction. Based on clinical observations, the disclosed population specific approach is able to account for underlying genetic mutations associated with a specific population, thereby allowing for improved prediction (e.g., stratification of patient risk) for a patient. The improved prediction may give health care professionals improved guidance on patient treatment. For example, the more accurate prediction may give health care professionals guidance on recommending that eligible cancer patients undergo treatment (e.g., radiation treatment, chemotherapy treatment, surgical resection, etc.) or not.

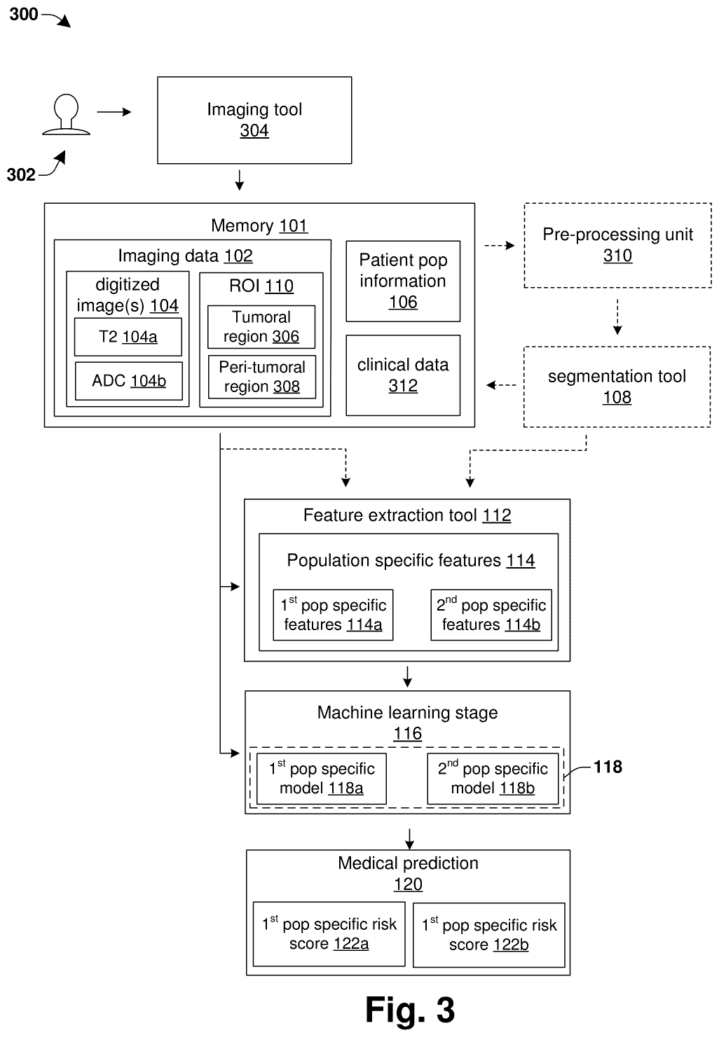

FIG. 3 illustrates some additional embodiments of a block diagram of a machine vision system 300 comprising population specific machine learning models configured to determine a population specific medical prediction for a cancer patient.

The machine vision system 300 comprises a memory 101 configured to store imaging data 102 comprising one or more digitized images 104 from one or more cancer patients 302. The one or more digitized images 104 within the imaging data 102 respectively have patient population information 106 associated with them. For example, a first digitized image may be associated with a patient of a first population, a second digitized image may be associated with a patient of a second population, etc. In some embodiments, the memory 101 may comprise electronic memory (e.g., solid state memory, SRAM (static random-access memory), DRAM (dynamic random-access memory), and/or the like).

In some embodiments, the imaging data 102 may comprise one or more radiological images obtained by operating an imaging tool 304 upon the one or more cancer patients 302. In various embodiments, the one or more radiological images may comprise a pre-treatment image. In some embodiments, the imaging tool 304 may comprise a multi-parametric MRI (magnetic resonance imaging) tool (e.g., a T2W MRI machine) configured to perform sequences that generate one or more of a T2-weighted (T2W) MRI image 104a, a diffusion weighted MRI image 104b, a dynamic contrast enhanced MRI image, and/or the like). For example, in some such embodiments, the T2W MRI image 104a may be obtained using a T2-weighted turbo spin echo sequence. In some embodiments, the one or more radiological images may comprise an apparent diffusion coefficient (ADC) map generated from the diffusion weighted MRI image 104b. In some embodiments, ROI within the T2W MRI image 104a may be mapped onto the diffusion weighted MRI image 104b via rigid registration followed by deformable co-registration. In other embodiments, the imaging tool 304 may comprise a CT (computed tomography) scanner, an x-ray machine, a PET (positron emission tomography) scanner, and/or the like.

In some embodiments, the imaging data 102 may comprise segmented imaging data that identifies one or more ROI 110. In some embodiments, the one or more ROI 110 may comprise a tumoral region 306 and a peri-tumoral region 308 (e.g., surrounding the tumoral region 306). In some embodiments, the peri-tumoral region 308 may be identified by extending a tumor boundary outwards for a predetermined distance (e.g., approximately 3 millimeters (mm), approximately 5 mm, etc.). In some embodiments, the one or more digitized images 104 may be provided to a segmentation tool 108 that is configured to segment the one or more digitized images 104 to identify the one or more ROI 110.

In some embodiments, the segmentation tool 108 may comprise a first machine learning model (e.g., a deep learning model) operated on one or more processors (e.g., one or more central processing units (CPUs), graphics processing units (GPUs), and/or the like). In other embodiments, the segmentation tool 108 may comprise a processor running a watershed algorithm, or the like. In some embodiments, the segmentation tool 108 may be configured to generate one or more binary masks having a value of “1” in image units (e.g., pixels, voxels, etc.) identified as being within the one or more ROI 110 and having a value of “0” in image units outside of the one or more ROI 110.

In some embodiments, the imaging data 102 may be provided to a pre-processing unit 310 prior to being provided to the segmentation tool 108. The pre-processing unit 310 may be configured to modify imaging intensities to enhance tissue specific meaning. For example, the pre-processing unit 310 may be configured to determine a standardized template distribution using a median image intensity template. Intensity distributions from each digitized image are then mapped to the standardized template distribution resulting in standardized images within a defined intensity range.

A feature extraction tool 112 is configured to extract a plurality of population specific features 114 from within the one or more ROI 110. In some embodiments, the plurality of population specific features 114 may comprise features that characterize a cancer heterogeneity. In some embodiments, the plurality of population specific features 114 may comprise one or more of first-order and second-order statistics (e.g., a mean, median, standard deviation, and range), Haralick features which are intensity distribution derived from gray-level-cooccurrence matrix (e.g., an entropy, energy, inertia, inverse difference moment, marginal distributions, correlation, information measures of correlation, sum of average, sum of variance, sum of entropy, difference of average, difference of variance, difference of entropy, and contrast) by varying a window size (e.g., 3 pixels×3 pixels, 5 pixels×5 pixels, 7 pixels×7 pixels), Gabor features, and CoLIAge features. In some embodiments, the feature extraction tool 112 may be implemented as computer code run by a processing unit (e.g., a central processing unit including one or more transistor devices configured to operate computer code to achieve a result, a microcontroller, or the like).

In various embodiments, the plurality of population specific features 114 may comprise a first plurality of population specific features 114a (e.g., African American specific features) and a second plurality of population specific features 114b (e.g., Caucasian American specific features). In some embodiments, the first plurality of population specific features 114a are features that are highly (e.g., most) prognostic of cancer stratification in African American patients and the second plurality of population specific features 114b are features that are highly (e.g., most) prognostic of cancer stratification in Caucasian American patients. For example, the first plurality of population specific features 114a may comprise first order texture features that capture spatial intensity relationships on the one or more digitized images 104 (e.g., on T2-weighted MRI and diffusion weighted MRI), while the second plurality of population specific features 114b may comprise intensity co-occurrence based radiomic features (e.g., Haralick features).

A machine learning stage 116 is configured to operate upon the population specific features 114 to generate a medical prediction 120. The machine learning stage 116 comprises a plurality of population specific machine learning models 118a-118n that have been trained to generate the medical prediction 120 for patients of a specific population. For example, a first population specific machine learning model 118a is configured to operate upon the first plurality of population specific features 114a to calculate a first population specific risk score 122a for a patient of a first population and a second population specific machine learning model 118n is configured to operate upon the second plurality of population specific features 114n to calculate a second population specific risk score 122b for a patient of a second population. In some embodiments, the medical prediction 120 may comprise a probability score indicating a likelihood of a presence of clinically significant cancer.

In some embodiments, the plurality of population specific machine learning models 118a-118n may comprise one or more of a regression model, a, a random forest model, a support vector machine, a linear discriminant analysis (LDA) classifier, a naÏve Bayes classifier, and/or the like. In some embodiments, the plurality of population specific machine learning models 118a-118n may be operated on one or more processors (e.g., one or more central processing units (CPUs), graphics processing units (GPUs), and/or the like).

In some embodiments, the plurality of population specific machine learning models 118a-118n may also be configured to include clinical data 312 related to the cancer patient 302. For example, clinical data 312 from the cancer patient 302 may be stored in the memory 101. The plurality of population specific machine learning models 118a-118n may be configured to receive the clinical data 312 from the memory 101 and to use the clinical data 312 as inputs to generate the medical prediction 120. In various embodiments, the clinical data 312 may include demographic information (e.g., age, sex, etc.), height, weight, body mass index, lab results, prostate specific antigen (PSA), tumor volume, PI-RADS v2.1, and/or the like, of the cancer patient 302.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

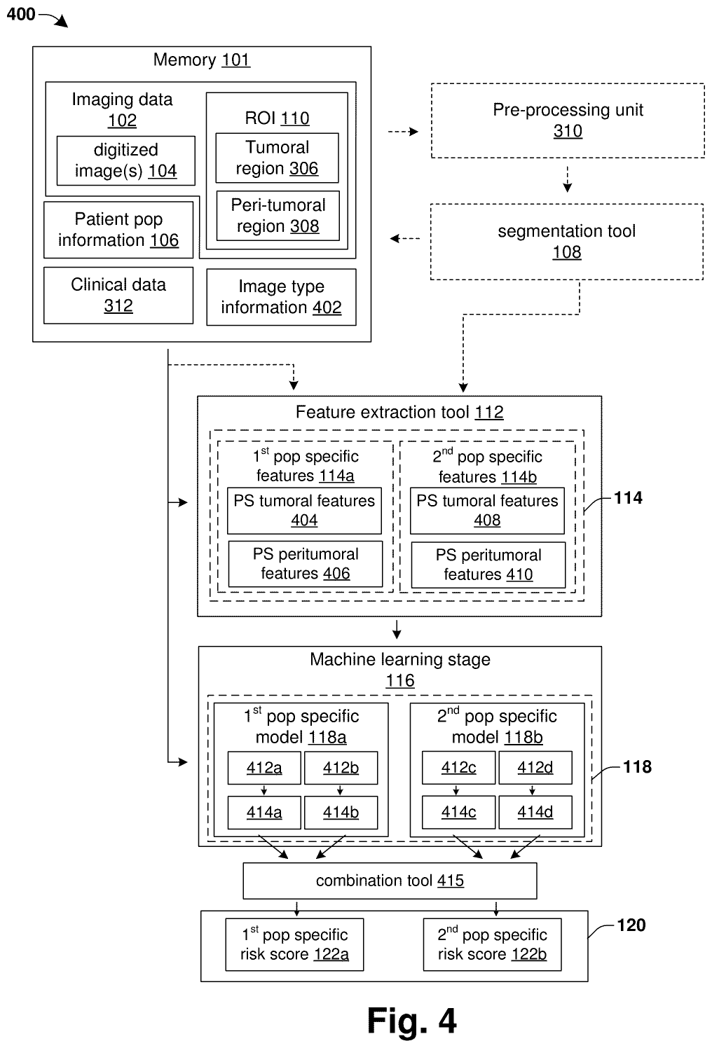

FIG. 4 illustrates some additional embodiments of a block diagram of a machine vision system 400 comprising population specific machine learning models configured to determine a population specific medical prediction for a cancer patient.

The machine vision system 400 comprises a memory 101 configured to store an imaging data 102 including one or more digitized images 104 from one or more cancer patients 302. In some embodiments, the imaging data 102 may comprise segmented imaging data that identifies one or more ROI 110. In some embodiments, the one or more ROI 110 may comprise a tumoral region 306 and a peri-tumoral region 308 (e.g., surrounding the tumoral region 306). The one or more digitized images 104 within the imaging data 102 respectively have patient population information 106 associated with them.

A feature extraction tool 112 is configured to extract a plurality of population specific features 114 from within the one or more ROI 110. The plurality of population specific features 114 may comprise a first plurality of population specific features 114a corresponding to a first population and a second plurality of population specific features 114b corresponding to a second population. It has been appreciated that different features may have prognostic value for different populations and/or for different ROI within a population. Therefore, in some embodiments, the plurality of population specific feature 114 may comprise different features for different ones of the ROI 110. For example, the first plurality of population specific features 114a may comprise a first plurality of population specific tumoral features 404 extracted from a tumoral region 306 of a first patient of a first population and a first plurality of population specific peri-tumoral features 406 extracted from a peri-tumoral region 308 of the first patient. The second plurality of population specific features 114b may comprise a second plurality of population specific tumoral features 408 extracted from a tumoral region 306 of a second patient of a second population and a second plurality of population specific peri-tumoral features 410 extracted from a peri-tumoral region 308 of the second patient. The first plurality of population specific tumoral features 404, the first plurality of population specific peri-tumoral features 406, the second plurality of population specific tumoral features 408, and the second plurality of population specific peri-tumoral features 410 may comprise one or more different features. In some embodiments, tumoral features may be extracted from 2D slice of T2W and ADC images within a cancer ROI and distribution statistics (e.g., mean, variance, skewness, and kurtosis) of each of the features over the entire ROI may be considered as feature vector for that lesion.

A machine learning stage 116 is configured to operate upon the population specific features 114 to generate a medical prediction 120. The machine learning stage 116 comprises a plurality of population specific machine learning models 118a-118b. In some embodiments, the plurality of population specific machine learning models 118a-118b may respectively comprise a tumoral machine learning model trained to generate a tumoral risk score using tumoral features and a peri-tumoral model trained to generate a peri-tumoral risk score using peri-tumoral features. The tumoral risk score and the peri-tumoral risk score are subsequently combined to form a population specific risk score.

For example, a first population specific machine learning model 118a may comprise a first population specific tumoral model 412a and a first population specific peri-tumoral model 412b. The first population specific tumoral model 412a is configured to operate on the first plurality of population specific tumoral features 404 to generate a first tumoral population specific risk score 414a and the first population specific peri-tumoral model 412b is configured to operate on the first plurality of population specific peri-tumoral features 406 to generate a first peri-tumoral population specific risk score 414b. A combination tool 415 is configured to combine the first tumoral population specific risk score 414a and the first peri-tumoral population specific risk score 414b to generate a first population specific risk score 122a. Similarly, a second population specific machine learning model 118b may comprise a second population specific tumoral model 412c and a second population specific peri-tumoral model 412d. The second population specific tumoral model 412c is configured to operate on the second plurality of population specific tumoral features 408 to generate a second tumoral population specific risk score 414c and the second population specific peri-tumoral model 412d is configured to operate on the second plurality of population specific peri-tumoral features 410 to generate a second peri-tumoral population specific risk score 414d. The combination tool 415 is further configured to combine the second tumoral population specific risk score 414c and the second peri-tumoral population specific risk score 414d to generate a second population specific risk score 122b.

In some additional embodiments, the feature extraction tool 112 and/or the plurality of population specific machine learning models 118 may also take into consideration a type of image. For example, the feature extraction tool 112 may extract different features from different imaging types. Furthermore, the plurality of population specific machine learning models 118 may comprise different models for different image type (e.g., a first population specific machine learning model for T2W MRI images, a second population specific machine learning model for ADC MRI images). In some embodiments, imaging information 402 corresponding to the one or more digitized images 104 may be stored in the memory 101 and accessed by the feature extraction tool 112 and/or the machine learning stage 116 to determine what features and/or machine learning models are to be used for an image.

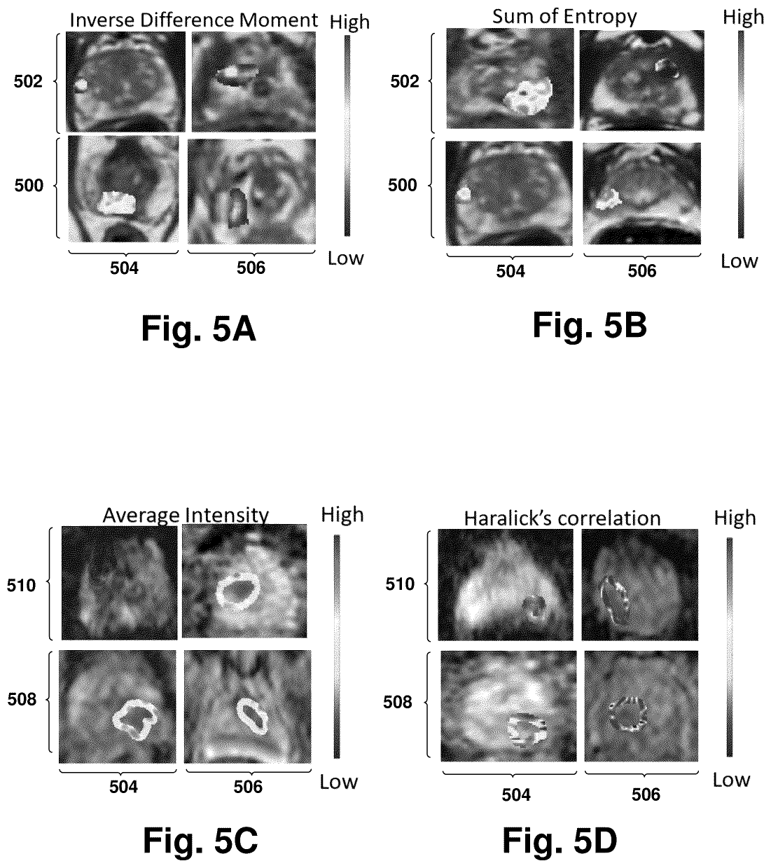

FIGS. 5A-5D illustrate exemplary images showing radiomic features within ROI corresponding to patients of different populations classified as having clinically significant cancer and clinically insignificant cancer.

FIGS. 5A-5B illustrate a first plurality of images 500 having an ROI comprising a tumoral region from patients with clinically significant cancer and a second plurality of images 502 having an ROI comprising a tumoral region from patients with clinically insignificant cancer. The first plurality of images 500 and the second plurality of images 502 include images 504 from an African American patient and images 506 from a Caucasian American patient.

As can be seen in FIG. 5A, a Haralick's inverse difference moment extracted from a tumoral region has different values that can differentiate between African American patients having clinically significant cancer and clinically insignificant cancer. However, a Haralick's inverse difference moment extracted from a tumoral region cannot meaningfully differentiate between Caucasian American patients having clinically significant cancer and clinically insignificant cancer.

As can be seen in FIG. 5B, a Haralick's sum of entropy extracted from a tumoral region has different values that can differentiate between Caucasian American patients having clinically significant cancer and clinically insignificant cancer. However, a Haralick's sum of entropy extracted from a tumoral region cannot meaningfully differentiate between African American patients having clinically significant cancer and clinically insignificant cancer.

FIGS. 5C-5D illustrate a first plurality of images 508 having an ROI comprising a peri-tumoral region from patients with clinically significant cancer and a second plurality of images 510 having an ROI comprising a peri-tumoral region from patients with clinically insignificant cancer. The first plurality of images 508 and the second plurality of images 510 include images 504 from an African American patient and images 506 from a Caucasian American patient.

As can be seen in FIG. 5C, an average intensity extracted from a peri-tumoral region can differentiate between African American patients having clinically significant cancer and clinically insignificant cancer. However, an average intensity extracted from a peri-tumoral region cannot meaningfully differentiate between Caucasian American patients having clinically significant cancer and clinically insignificant cancer.

As can be seen in FIG. 5D, a Haralick's correlation can differentiate between Caucasian American patients having clinically significant cancer and clinically insignificant cancer. However, a Haralick's correlation cannot meaningfully differentiate between African American patients having clinically significant cancer and clinically insignificant cancer.

Therefore, as can be seen in FIGS. 5A-5D, radiomic features associated with clinically significant cancer are differentially expressed between population groups, such that different radiomic features may have prognostic value for different populations and/or for different ROI within a population. These different radiomic features allow for the disclosed machine vision system to accurately assess differential heterogeneity associated with clinically significant cancer (e.g., prostate cancer in men, ovarian cancer in women, etc.) between different patient populations.

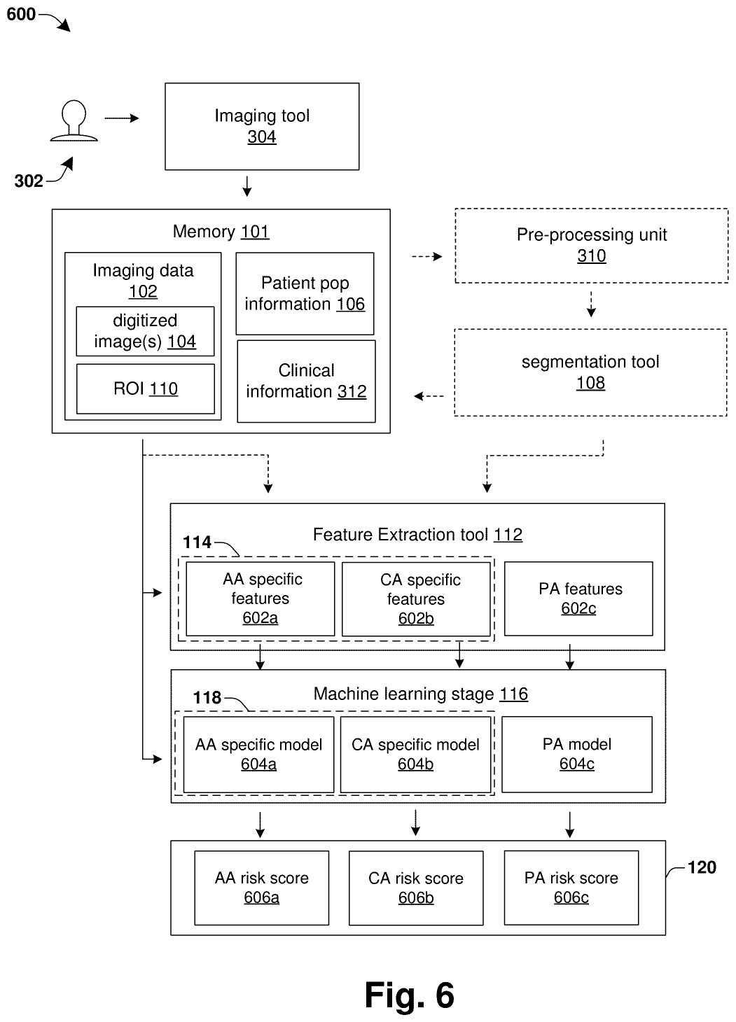

FIG. 6 illustrates some additional embodiments of a block diagram of a prostate cancer assessment system 600 comprising population specific machine learning models configured to determine a population specific risk score for a prostate cancer patient.

The prostate cancer assessment system 600 comprises a memory 101 configured to store imaging data 102 comprising one or more digitized images 104 from one or more cancer patients 302. In some embodiments, the one or more digitized images 104 may comprise a first radiological image of an African American prostate cancer patient and a second radiological image of a Caucasian American prostate cancer patient. In some embodiments, the one or more digitized images 104 may comprise segmented images having one or more ROI 110. The one or more digitized images 104 within the imaging data 102 respectively have patient population information 106 associated with them.

A feature extraction tool 112 is configured to extract a plurality of population specific features 114 from the ROI 110. In some embodiments, the plurality of population specific features 114 may comprise a plurality of African American specific features 602a or a plurality of Caucasian American specific features 602b. The plurality of African American specific features 602a are features that are highly (e.g., most) prognostic of prostate cancer stratification in African American patients. For example, the plurality of African American specific features 602a may comprise first order texture features that capture spatial intensity relationships on the one or more digitized images 104 (e.g., on T2-weighted MRI and diffusion weighted MRI). The plurality of Caucasian American specific features 602b are features that are highly (e.g., most) prognostic of prostate cancer stratification in Caucasian American patients. For example, the plurality of Caucasian American specific features 602b may comprise intensity co-occurrence based radiomic features (e.g., Haralick features).

In some additional embodiments, the feature extraction tool 112 is further configured to extract a plurality of a population agnostic features 602c. The plurality of population agnostic features 602c comprise features that are highly (e.g., most) prognostic of prostate cancer stratification in patients not having a specific population (e.g., features identified using a group of patients having different population).

A machine learning stage 116 is configured to access the plurality of population specific features 114 and/or the population agnostic features 602c. The machine learning stage 116 comprises an African American specific machine learning model 604a configured to operate on the plurality of African American specific features 602a to generate an African American specific risk score 606a that is indicative of prostate cancer risk stratification for an African American patient. The machine learning stage 116 further comprises a Caucasian American specific machine learning model 604b configured to operate on the plurality of Caucasian American specific features 602b to generate a Caucasian American specific risk score 606b that is indicative of prostate cancer risk stratification for a Caucasian American patient. In some embodiments, the machine learning stage 116 further comprises a population agnostic machine learning model 604c configured to operate on the plurality of population agnostic features 602c to generate a population agnostic risk score 606c that is indicative of prostate cancer risk stratification for a patient of unidentified population.

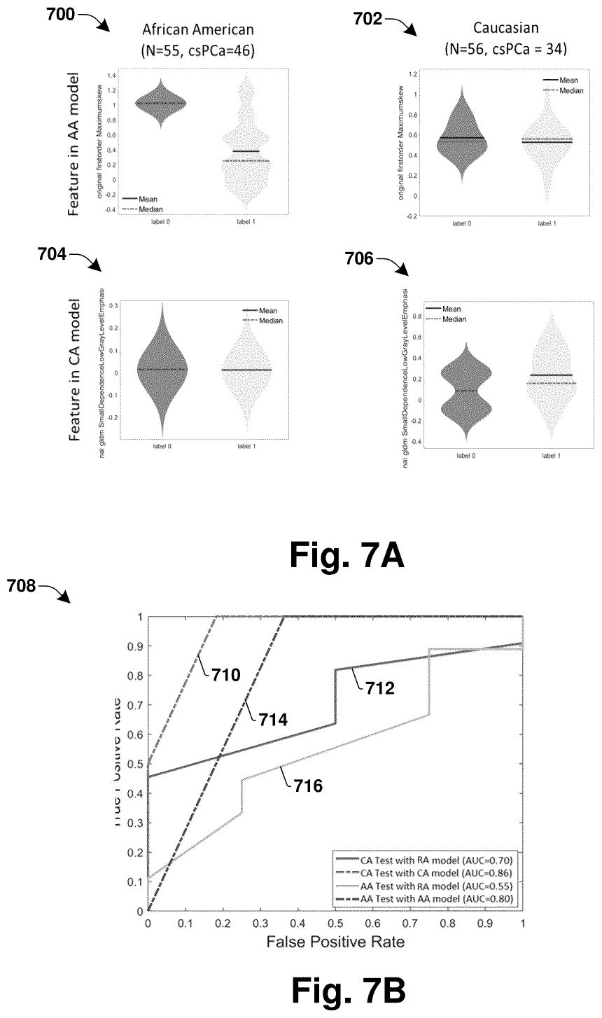

FIG. 7A shows exemplary violin plots of radiomic features from MRI scans associated with clinically significant prostate cancer in African American patients and Caucasian American patients.

FIG. 7A illustrates a first pair of violin plots 700-702 corresponding to a first radiomic feature and a second pair of violin plots 704-706 corresponding to a second radiomic feature. As shown in the first pair of violin plots 700-702, the first radiomic feature is prognostic of cancer risk stratification in African American patients (plot 700), but not in Caucasian American patients (plot 702). As shown in the second pair of violin plots 704-706, the second radiomic feature is not prognostic of cancer risk stratification in African American patients (plot 704), but is in Caucasian American patients (plot 706).

Therefore, the violin plots 700-706 shown in FIG. 7A illustrate that different radiomic features are prognostic for different population groups (e.g., African American patients and Caucasian American patients), thereby indicating that the use of population specific machine learning features and models may be prognostic for prostate cancer assessment.

FIG. 7B shows a graph 708 illustrating exemplary receiver operating characteristic (ROC) curves demonstrating the classification performance of population specific machine learning models for African American patients and Caucasian American patients.

As can be seen in graph 708, a Caucasian American (CA) specific machine learning model trained using Caucasian American specific features is able to achieve a ROC curve 710 having a higher area under curve (AUC) than a population agnostic (RA) specific machine learning model trained using Caucasian American specific features (ROC curve 712). For example, a Caucasian American specific machine learning model trained using Caucasian American specific features is able to achieve an AUC of 0.86, while a population agnostic specific machine learning model trained using Caucasian American specific features is able to achieve an AUC of 0.70.

Similarly, an African American (AA) specific machine learning model trained using African American specific features is able to achieve a ROC curve 714 having a higher AUC than a population agnostic specific machine learning model trained using African American specific features (ROC curve 716). For example, an African American specific machine learning model trained using African American specific features is able to achieve an AUC of 0.80, while a population agnostic specific machine learning model trained using African American specific features is able to achieve an AUC of 0.55.

The higher AUC achieved by the population specific models (e.g., CA specific machine learning model and AA specific machine learning model) shown in graph 708 illustrates the ability of the disclosed prostate cancer assessment system to achieve an accurate stratification of prostate cancer risk using population specific features and population specific machine learning models.

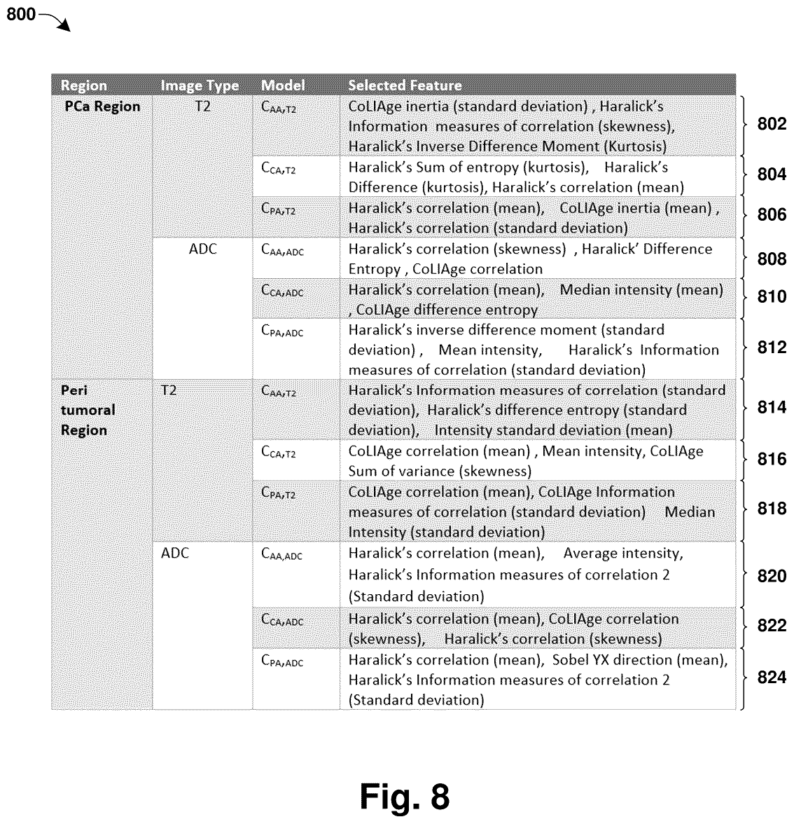

FIG. 8 illustrates a table 800 showing some exemplary radiomic features that may be extracted from tumoral regions and peri-tumoral regions of images from a cancer patient. It will be appreciated that the radiomic features listed in table 800 are not limiting of the radiomic features that may be used by the disclosed apparatus, but rather are only example radiomic features that may be used.

The table 800 shows features that may be prognostic for T2-weighted (T2) MRI images and for apparent diffusion coefficient (ADC) MRI images in both tumoral regions and peri-tumoral regions. For each of the T2 MRI images and ADC MRI images, different features are prognostic for different models corresponding to different populations. For example, in the tumoral region of a T2 MRI image, a first set of features 802 are prognostic for an African American specific model, a second set of features 804 are prognostic for a Caucasian American model, and a third set of features 806 are prognostic for a population agnostic model. In the tumoral region of an ADC MRI image, a first set of features 808 are prognostic for an African American specific model, a second set of features 810 are prognostic for a Caucasian American model, and a third set of features 812 are prognostic for a population agnostic model. In the peri-tumoral region of a T2 MRI image, a first set of features 814 are prognostic for an African American specific model, a second set of features 816 are prognostic for a Caucasian American model, and a third set of features 818 are prognostic for a population agnostic model. In the peri-tumoral region of an ADC MRI image, a first set of features 820 are prognostic for an African American specific model, a second set of features 822 are prognostic for a Caucasian American model, and a third set of features 824 are prognostic for a population agnostic model.

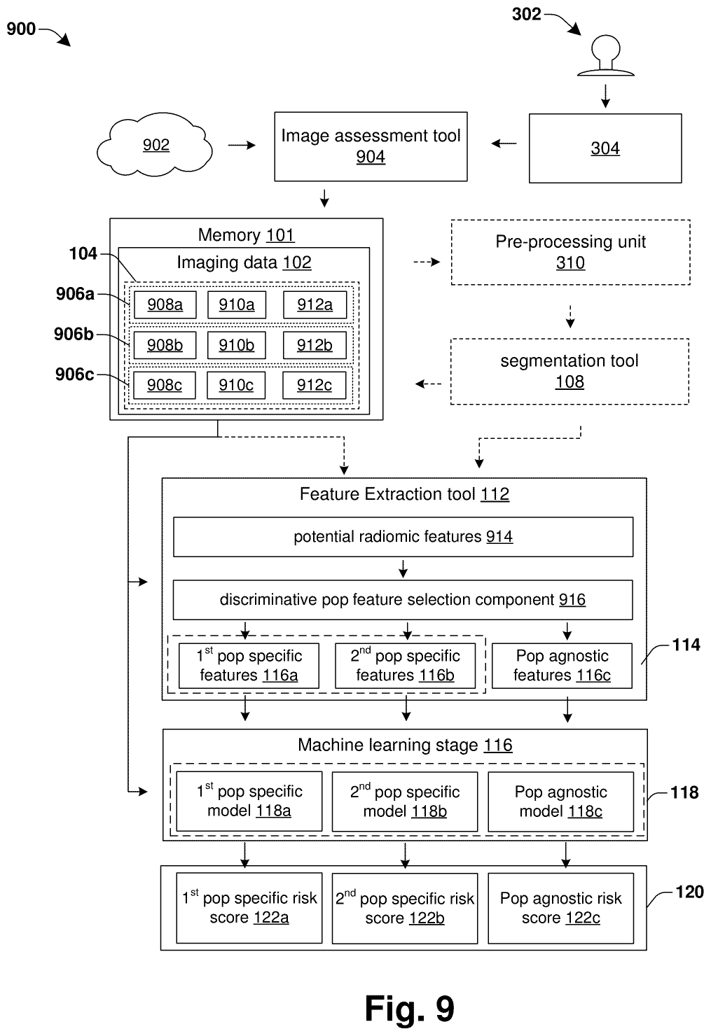

FIG. 9 illustrates a block diagram of some additional embodiments of a machine vision system 900 comprising population specific machine learning models that are configured to be trained to determine a medical prediction for a patient using population specific features extracted from digitized images.

The machine vision system 900 comprises a memory 101 configured to store imaging data 102 including digitized images 104 from a plurality of cancer patients 302 (e.g., prostate cancer patients). In some embodiments, the imaging data 102 comprises MRI images of prostate tissue. In various embodiments, the imaging data 102 may be obtained by an imaging tool 304 and/or from an on-line database 902 and/or archive containing plurality of radiological images from cancer patients generated at different sites (e.g., different hospitals, research laboratories, and/or the like). Prior to including radiological images within the imaging data 102, the radiological images may be subjected to an image assessment tool 904 configured to apply one or more inclusion criteria and exclusion criteria.

The imaging data 102 may include a first population specific (e.g., African American) training data set 906a, a second population specific (e.g., Caucasian American) training data set 906b, and a population agnostic training data set 906c. The first population specific training data set 906a comprises radiological images of patients of a first population and not of a second population (e.g., of African American patients and not of Caucasian American patients). The second population specific training data set 906b comprises radiological images of patients of a second population and not of a first population (e.g., of Caucasian American patients and not of African American patients). The population agnostic training data set 906c comprises radiological images from patients of both the first population and the second population (e.g., of African American patients and Caucasian American patients). In some embodiments, the first population specific training data set 906a may comprise training data 908a, testing data 910a, and/or validation data 912a. In some embodiments, the second population specific training data set 906b may comprise training data 908b, testing data 910b, and/or validation data 912b. In some embodiments, the population agnostic training data set 906c may comprise training data 908c, testing data 910c, and/or validation data 912c.

A segmentation tool 108 is configured to segment the digitized images 104 to identify one or more regions of interest (ROI). A feature extraction tool 112 is configured to extract a plurality of potential radiomic features 914 associated with the one or more ROI. In some embodiments, the plurality of potential radiomic features 914 may characterize prostate cancer heterogeneity. In some embodiments, the plurality of potential radiomic features 914 may include one or more of first and second order statistics, Haralick features quantifying intensity co-occurrences, Gabor features quantifying texture frequency across specific directions, and the like. In some embodiments, the feature extraction tool 112 may be trained using the first population specific training data set 906a, the second population specific training data set 906b, and the population agnostic training data set 906c.

A discriminative feature selection component 916 may be operated upon the plurality of potential radiomic features 914 to identify a first plurality of population specific features 116a that are highly (e.g., most) prognostic of clinically significant cancer in patients of a first population (e.g., African American patients), to identify a second plurality of population specific features 116b that are highly (e.g., most) prognostic of clinically significant cancer in patients of a second population (e.g., Caucasian American patients), and/or to identify a plurality of population agnostic features 116c that are highly (e.g., most) correlated with clinically significant cancer. The discriminative feature selection component 916 may comprise a Wilcoxon rank-sum test (e.g., that identifies prognostic features as having a p-value of less than or equal to approximately 0.05). In other embodiments, the discriminative feature selection component 916 may utilize a Pearsons's correlation coefficient set at a predetermined threshold (e.g., of 0.9, of 0.8, etc.), a maximum relevance and minimum redundancy (mRMR) feature selection method, and/or the like.

A first population specific machine learning model 118a (e.g., an African American specific machine learning model) is configured to act upon the first plurality of population specific features 116a to generate a first population specific risk score 122a (e.g., an African American specific risk score). In some embodiments, the training data 908a may be used to train initial versions of the first population specific machine learning model 118a. The initial versions of the first population specific machine learning model 118a may be subsequently fine-tuned using the testing data 910a to generate one or more evaluation models. The validation data 912a may then be used to generate the first population specific machine learning model 118a from the one or more evaluation models. In some embodiments, the first population specific machine learning model 118a may comprise a support vector machine learning classifier. In some embodiments, a 3-fold cross validation framework may be used during training of the first population specific machine learning model 118a.

A second population specific machine learning model 118b (e.g., a Caucasian American specific machine learning model) is configured to act upon the second plurality of population specific features 116b to generate a second population specific risk score 122b (e.g., Caucasian American specific risk score). The training data 908b may be used to train initial versions of the second population specific machine learning model 118b. The initial versions of the second population specific machine learning model 118b may be subsequently fine-tuned using the testing data 910b to generate one or more evaluation models. The validation data 912b may then be used to generate the second population specific machine learning model 118b from the one or more evaluation models. In some embodiments, the second population specific machine learning model 118b may comprise a support vector machine learning classifier. In some embodiments, a 3-fold cross validation framework may be used during training of the second population specific machine learning model 118b.

A population agnostic machine learning model 118c is configured to act upon the plurality of population agnostic features 116c to generate a population agnostic risk score 122c. The training data 908c may be used to train initial versions of the population agnostic machine learning model 118c. The initial versions of the population agnostic machine learning model 118c may be subsequently fine-tuned using the testing data 910c to generate one or more evaluation models. The validation data 912c may then be used to generate the population agnostic machine learning model 118c from the one or more evaluation models. In some embodiments, the population agnostic machine learning model 118c may comprise a support vector machine learning classifier. In some embodiments, a 3-fold cross validation framework may be used during training of the population agnostic machine learning model 118c.

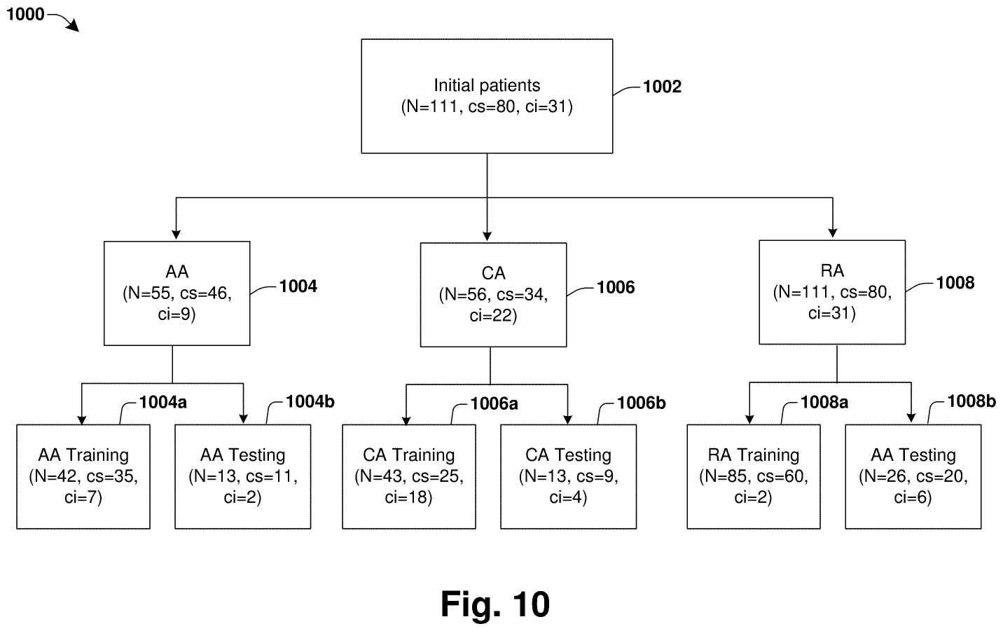

FIG. 10 illustrates a block diagram 1000 showing an example patient selection (e.g., as performed by image assessment tool 904 of FIG. 9) for a method of training a disclosed machine vision system.

As shown in block diagram 1000, an initial group of patients 1002 is provided. The initial group of patients 1002 has a first number of patients, including clinically significant (cs) cases (e.g., patients having a significant risk of prostate cancer) and clinically insignificant (ci) cases (e.g., patients not having a significant risk of prostate cancer). For example, in some embodiments the initial group of patients 1002 may comprise 111 patients including 80 that are clinically significant cases and 31 that are clinically insignificant cases.

The initial group of patients 1002 may be split into an African American (AA) group 1004, a Caucasian American (CA) group 1006, and a population agnostic (RA) group 1008 including both African Americans and Caucasian Americans. The African American (AA) group 1004 is further divided into a training set 1004a and a testing set 1004b. The Caucasian American (CA) group 1006 is further divided into a training set 1006a and a testing set 1006b. The population agnostic (RA) group 1008 is further divided into a training set 1008a and a testing set 1008b.

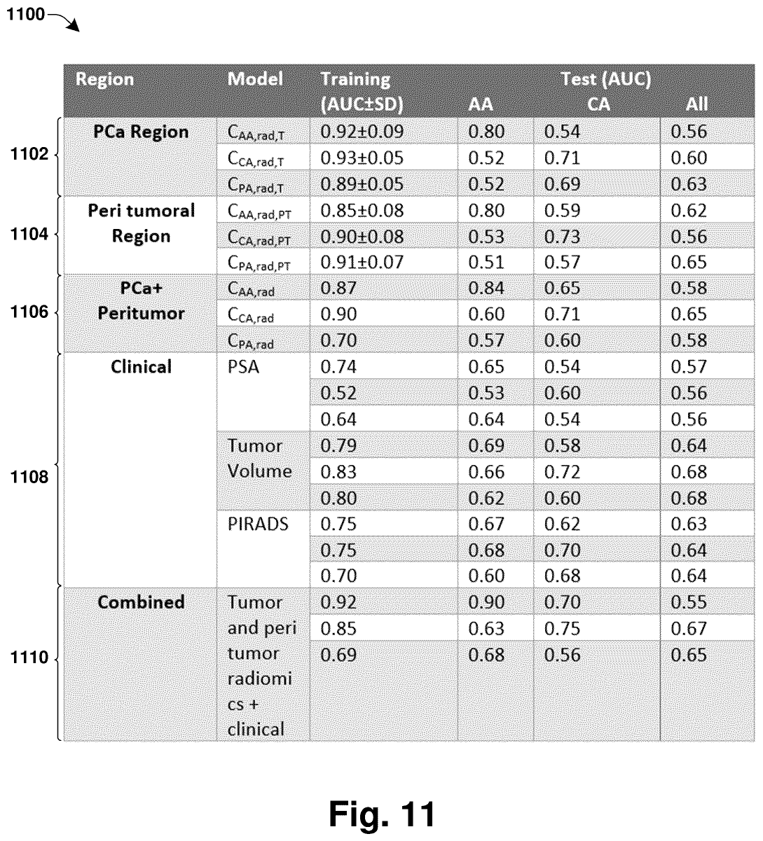

FIG. 11 illustrates a table 1100 showing some exemplary performance metrics achieved by different biomarkers used to predict prostate cancer risk.

Table 1100 shows performance evaluation of machine vision systems operating upon population specific models (AA and CA) and population agnostic (PA) models using radiomic features extracted from tumoral regions 1102, using radiomic features extracted from peri-tumoral regions 1104, using radiomic features extracted from tumoral and peri-tumoral regions 1106, using clinical variables 1108, and using a combination of radiomic features extracted from tumoral region, radiomic features extracted from peri-tumoral regions, and clinical variables 1110.

As can be seen in table 1100, machine vision systems operating upon radiomic features with a population specific approach (AA and CA) models demonstrated significant improvement over population agnostic (PA) models. Furthermore, the combination of radiomic features and clinical variables was able to achieve further improvement over the use of radiomic features alone.

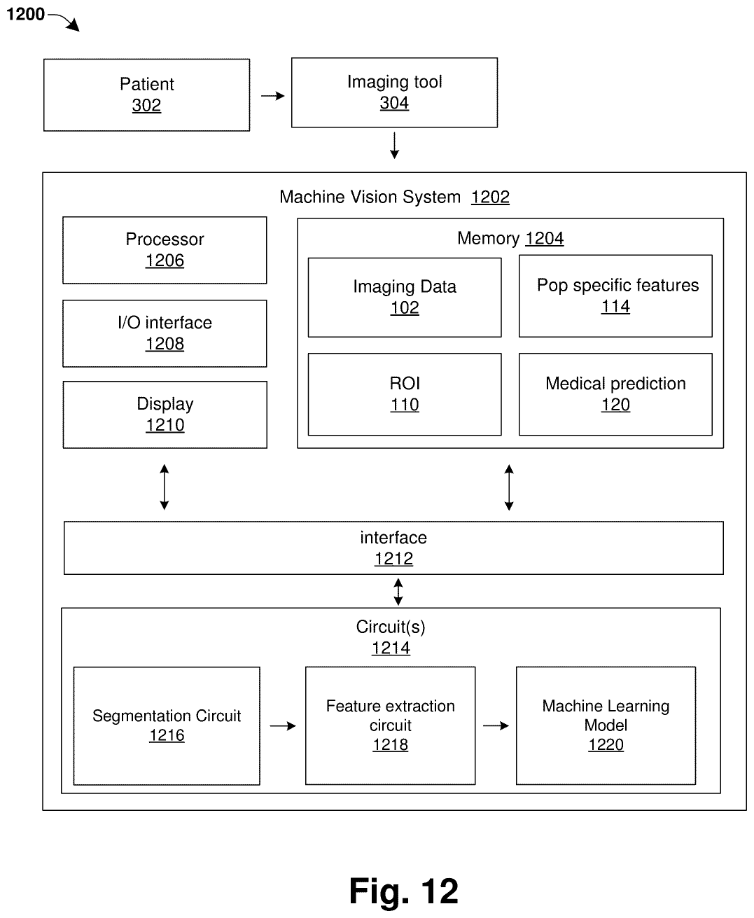

FIG. 12 illustrates a block diagram of some embodiments of a prognostic apparatus 1200 comprising a disclosed machine vision system configured to use population specific machine learning models to generate a medical prediction.

The prognostic apparatus 1200 comprises a machine vision system 1202. The machine vision system 1202 is coupled to an imaging tool 304 (e.g., an MRI scanner) that is configured to generate one or more radiological images (e.g., MRI scans) corresponding to a cancer patient 302.

The machine vision system 1202 comprises a processor 1206 and a memory 1204. The processor 1206 can, in various embodiments, comprise circuitry such as, but not limited to, one or more single-core or multi-core processors. The processor 1206 can include any combination of general-purpose processors and dedicated processors (e.g., graphics processors, application processors, etc.). The processor 1206 can be coupled with and/or can comprise memory (e.g., memory 1204) or storage and can be configured to execute instructions stored in the memory 1204 or storage to enable various apparatus, applications, or operating systems to perform operations and/or methods discussed herein.

Memory 1204 can be further configured to store imaging data 102 comprising the one or more radiological images (e.g., MRI scans). In some embodiments, the one or more radiological images may comprise one or more multi-parametric MRI images, which respectively include a T2-weighted (T2W) Magnetic Resonance Imaging (MRI) image, a diffusion weighted MRI image, or a dynamic contrast enhanced MRI image. The one or more radiological images include a plurality of imaging units (e.g., pixels, voxels, etc.) respectively having an associated intensity pixels. In some additional embodiments, the one or more radiological images may be stored in the memory 1204 as one or more training sets, testing sets, and/or validation sets of radiological images for training a machine learning circuit.

The machine vision system 1202 also comprises an input/output (I/O) interface 1208 (e.g., associated with one or more I/O devices), a display 1210, one or more circuits 1214, and an interface 1212 that connects the processor 1206, the memory 1204, the I/O interface 1208, the display 1210, and the one or more circuits 1214. The I/O interface 1208 can be configured to transfer data between the memory 1204, the processor 1206, the one or more circuits 1214, and external devices (e.g., imaging tool 304).

In some embodiments, the one or more circuits 1214 may comprise hardware components. In other embodiments, the one or more circuits 1214 may comprise software components. In such embodiments, the one or more circuits 1214 may execute code stored in the memory 1204. The one or more circuits 1214 can comprise a segmentation circuit 1216 configured to segment respective ones of the one or more radiological images to identify one or more regions of interest (ROI) 110. In some embodiments, the segmentation circuit 1216 may comprise a deep learning model/circuit.

In some additional embodiments, the one or more circuits 1214 may further comprise a feature extraction circuit 1218. In some embodiments, the feature extraction circuit 1218 is configured to extract a plurality of population specific features associated with the one or more ROI.

In some additional embodiments, the one or more circuits 1214 may further comprise a population specific machine learning circuit 1220. In some embodiments, the population specific machine learning circuit 1220 is configured to utilize the plurality of population specific features 114 to generate a medical prediction 120.

Although the medical prediction 120 is described herein as relating to cancer prediction for cancer patients, it will be appreciated that the disclosed machine vision system is not limited to such applications. Rather, in other embodiments, the disclosed machine vision system may be configured to generate a population specific medical prediction relating to other applications (e.g., cancer treatment response in cancer patients, allograft rejection in transplant patients, heart transplant rejection in heart transplant patients, etc.).

### Example Use Case 1

Background: African American (AA) men with prostate cancer (PCa) have 2.5 times higher risk of mortality compared to Caucasian American (CA) men. AA men tend to harbor genetic mutations associated with high risk PCa suggesting the biological basis of health disparities. Artificial Intelligence (AI) models in conjunction with prostate Magnetic Resonance Imaging (MRI) may result in improved, non-invasive PCa risk stratification. However, such models have not accounted for disparities between AA and CA men. We have appreciated that quantifying population specific PCa heterogeneity will result in improved risk stratification of AA men.

Objective: Our objective in this study is to train and validate population specific AI models for PCa risk stratification using screening MRI in AA men.

Methods: In this HIPAA compliant, IRB approved, retrospective study, we identified N=111 PCa patients (AA=55, CA=56, based on self-reported race) within our institutional cohort. N=86 (AA=42, CA=43) studies were used for training (DT) and N=26 (AA=13, CA=13) were used for hold-out validation (DV). All patients underwent 3 Telsa (3T) MRI with a pelvic phased array surface coil prior to biopsy. An experienced radiologist delineated PCa regions of interest (ROIs) on T2-weighted (T2W) and apparent diffusion coefficient (ADC) MRI sequences. Radiomic features were derived from ROls on T2W, ADC to characterize PCa heterogeneity, including first and second order statistics, Haralick features quantifying intensity co-occurrences, Gabor features quantifying texture frequency across specific directions. Wilcoxon rank-sum test was used to identify radiomic features associated (p<0.05) with clinically significant PCa (csPCa: biopsy Gleason Grade >1) separately within AA, CA and race agnostic (RA=CA+AA)) cohorts. These features in conjunction with support vector machine learning classifier were used to train models CAA, CCA and CRA corresponding to AA, CA and RA(=AA+CA) populations using a 3-fold cross validation framework. These were evaluated on DV in terms of AUC.

Results: First order texture features were associated with csPCa in AA men, while Haralick features were associated with csPCa in CA men. On DV, CAA, CCA resulted in AUC of 0.80 and 0.86 respectively while the race agnostic model CRA resulted in AUC=0.55 on AA cohort.

Conclusions: These preliminary results indicate that accounting for population specific PCa heterogeneity on MRI using AI may result in improved PCa risk stratification in AA men.

Therefore, the present disclosure relates to a method and associated apparatus for classifying tissue from a cancer patient using population specific radiomic features and a population specific machine learning classifier. The disclosed method and associated apparatus are able to achieve an improved performance over traditional manual assessments because the population specific radiomic features that the disclosed method and apparatus extracts from the digitized images are at a higher order or higher level than a human can resolve in the human mind or with pencil and paper.

In some embodiments, the present disclosure relates to a method. The method includes accessing one or more digitized images of a cancer patient of a first population; identifying one or more regions of interest within the one or more digitized images; extracting a plurality of population specific features from the one or more regions of interest within the one or more digitized images, the plurality of population specific features being features that have been identified as being prognostic of an outcome for patients of the first population; and operating a population specific machine learning model upon the plurality of population specific features to generate a medical prediction relating to the outcome.

In other embodiments, the present disclosure relates to a non-transitory computer-readable medium storing computer-executable instructions that, when executed, cause a processor to perform operations, including accessing a radiological image of an African American prostate cancer patient; extracting a plurality of African American specific features from the radiological image, the plurality of African American specific features being highly prognostic in stratifying prostate cancer risk in African American patients; and operating upon the plurality of African American specific features with an African American specific machine learning model to generate an African American specific risk score.

In yet other embodiments, the present disclosure relates to machine vision system including a memory configured to store a first digitized image of a cancer patient of a first population or a second digitized image of a cancer patient of a second population; a feature extraction tool configured to extract a plurality of first population specific features from the first digitized image or to extract a plurality of second population specific features from the second digitized image; and a machine learning stage having a first population specific machine learning model and a second population specific machine learning model, the first population specific machine learning model being configured to operate upon the plurality of first population specific features to generate a first population specific risk score and the second population specific machine learning model being configured to operate upon the plurality of second population specific features to generate a second population specific risk score.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

Embodiments discussed herein relate to training and/or employing machine learning models (e.g., unsupervised (e.g., clustering) or supervised (e.g., classifiers, etc.) models) to determine a medical prediction based on a combination of radiomic features and deep learning, based at least in part on features of medical imaging scans (e.g., MRI, CT, etc.) that are not perceivable by the human eye, and involve computation that cannot be practically performed in the human mind. As one example, machine learning classifiers and/or deep learning models as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

Examples herein can include subject matter such as an apparatus, including a digital whole slide scanner, an MRI system,, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing one or more digitized images of a cancer patient of a first population;
identifying one or more regions of interest within the one or more digitized images;
extracting a plurality of population specific features from the one or more regions of interest within the one or more digitized images, wherein the plurality of population specific features are features that have been identified as being prognostic of an outcome for patients of the first population; and
operating a population specific machine learning model upon the plurality of population specific features to generate a medical prediction relating to the outcome.

2. The method of claim 1, wherein the plurality of population specific features characterize prostate cancer heterogeneity within the one or more regions of interest.

3. The method of claim 1, wherein the first population is African American, the population specific features are African American specific features, and the population specific machine learning model is an African American specific machine learning model.

4. The method of claim 3, wherein the plurality of population specific features include first order texture features.

5. The method of claim 1, further comprising:
accessing an additional digitized image of an additional cancer patient, wherein the additional cancer patient is of a second population that is different than the first population;
identifying one or more additional regions of interest within the additional digitized image;
extracting a plurality of additional population specific features from the one or more additional regions of interest within the additional digitized image, wherein the plurality of additional population specific features are different than the plurality of population specific features; and
operating upon the plurality of additional population specific features with an additional population specific machine learning model to generate an additional population specific risk score, wherein the population specific machine learning model is different than the additional population specific machine learning model.

6. The method of claim 5, wherein the first population includes a self-reported race or a genetic ancestry of the cancer patient.

7. The method of claim 1, further comprising:
extracting a plurality of additional population specific features from an additional digitized image;
operating upon the plurality of additional population specific features with an additional population specific machine learning model to generate an additional population specific risk score;
wherein the plurality of population specific features are specific to African American patients and the plurality of additional population specific features are specific to Caucasian American patients; and
wherein the population specific machine learning model is configured to achieve a higher area under curve (AUC) using the plurality of population specific features than using the plurality of additional population specific features.

8. The method of claim 1, further comprising:
extracting a plurality of potential radiomic features associated with the one or more regions of interest from the one or more digitized images;
identifying a plurality of African American specific features from the plurality of potential radiomic features, wherein the plurality of African American specific features are highly prognostic of cancer risk stratification in African American cancer patients; and
identifying a plurality of Caucasian American specific features from the plurality of potential radiomic features, wherein the plurality of Caucasian American specific features are highly prognostic of cancer risk stratification in Caucasian American cancer patients.

9. The method of claim 1, further comprising:
operating the population specific machine learning model upon clinical data and the plurality of population specific features to generate the medical prediction.

10. The method of claim 1,
wherein the one or more regions of interest comprise a tumoral region and a peri-tumoral region; and
wherein the plurality of population specific features are extracted from the tumoral region and the peri-tumoral region.

11. A non-transitory computer-readable medium storing computer-executable instructions that, when executed, cause a processor to perform operations, comprising:
accessing a radiological image of an African American prostate cancer patient;
extracting a plurality of African American specific features from the radiological image, wherein the plurality of African American specific features are highly prognostic in stratifying prostate cancer risk in African American patients; and
operating upon the plurality of African American specific features with an African American specific machine learning model to generate an African American specific risk score.

12. The non-transitory computer-readable medium of claim 11, wherein the radiological image comprises a multi-parametric MRI image including a T2-weighted (T2W) Magnetic Resonance Imaging (MRI) image, a diffusion weighted MRI image, or a dynamic contrast enhanced MRI image.

13. The non-transitory computer-readable medium of claim 11, wherein the plurality of African American specific features include first order texture features that capture spatial intensity relationships on the radiological image.

14. The non-transitory computer-readable medium of claim 11, wherein the operations further comprise:
accessing an additional radiological image of a Caucasian American prostate cancer patient;
extracting a plurality of Caucasian American specific features from the additional radiological image, wherein the plurality of Caucasian American specific features are different than the plurality of African American specific features; and
operating upon the plurality of Caucasian American specific features with Caucasian American specific machine learning model to generate a Caucasian American specific medical prediction.

15. The non-transitory computer-readable medium of claim 11, wherein the African American specific risk score is indicative of a probability that the African American prostate cancer patient has clinically significant prostate cancer.

16. A machine vision system, comprising:
a memory configured to store a first digitized image of a cancer patient of a first population or a second digitized image of a cancer patient of a second population;
a feature extraction tool configured to extract a plurality of first population specific features from the first digitized image or to extract a plurality of second population specific features from the second digitized image; and
a machine learning stage comprising a first population specific machine learning model and a second population specific machine learning model, wherein the first population specific machine learning model is configured to operate upon the plurality of first population specific features to generate a first population specific risk score and the second population specific machine learning model is configured to operate upon the plurality of second population specific features to generate a second population specific risk score.

17. The machine vision system of claim 16, wherein the plurality of first population specific features are prognostic of cancer stratification in cancer patients of the first population and the plurality of second population specific features are prognostic of cancer stratification in cancer patients of the second population.

18. The machine vision system of claim 16, wherein the machine learning stage further comprises:
a population agnostic machine learning model configured to operate upon a plurality of population agnostic features that are prognostic of cancer stratification in a group of cancer patients having more than one population.

19. The machine vision system of claim 16,
wherein the plurality of first population specific features comprise a first plurality of population specific tumoral features and a first plurality of population specific peri-tumoral features;
wherein the first population specific machine learning model comprises a first population specific tumoral model configured to operate upon the first plurality of population specific tumoral features to generate a first tumoral population specific risk score; and
wherein the first population specific machine learning model further comprises a first population specific peri-tumoral model configured to operate upon the first plurality of population specific peri-tumoral features to generate a first peri-tumoral population specific risk score.

20. The machine vision system of claim 19, further comprising:
a combination tool configured to combine the first tumoral population specific risk score and the first peri-tumoral population specific risk score to generate the first population specific risk score.
